(19) United States
(12) Patent Application Publication
(10) Pub. No.: US 2021/0244432 A1
(43) Pub. Date: Aug. 12, 2021

(54) SKIN CONSTRUCT TRANSFER SYSTEM AND METHOD

(71) Applicant: The General Hospital Corporation, Boston, MA (US)

(72) Inventors: Richard Rox Anderson, Boston, MA (US); Charles R. Sperry, Springfield, VT (US); Alexander G. Sperry, Boston, MA (US); Joshua Tam, Andover, MA (US); Karl Wassmann, Boston, MA (US)

(21) Appl. No.: 17/251,598

(22) PCT Filed: Jun. 11, 2019
(86) PCT No.: PCT/US2019/036606

§ 371 Date: Dec. 11, 2020

Related U.S. Application Data
(60) Provisional application No. 62/683,284, filed on Jun. 11, 2018.
(60) Provisional application No. 62/758,138, filed on Nov. 9, 2018.

(51) Int. Cl. A61B 17/322; A61B 17/3205

(52) U.S. Cl. CPC A61B 17/322; A61B 2017/00747; A61B 17/32053

(57) ABSTRACT

Systems and methods for assembling a plurality of tissue grafts are provided. A system includes a pick and place unit, a transfer tool, and a control unit. The pick and place unit includes a moveable arm and the transfer tool is coupled to a distal end of the arm. The control unit is configured to control the moveable arm to move the transfer tool to a donor site and a graft construction site. The control unit is further configured to control the transfer tool to harvest the micro tissue grafts from the donor site and arrange the micro tissue grafts into a supportive matrix to form a custom graft.

### CROSS-REFERENCES TO RELATED APPLICATIONS

The present application is based on and claims priority to U.S. Provisional Patent Application No. 62/683,284, filed on Jun. 11, 2018, and U.S. Provisional Patent Application No. 62/758,138, filed on Nov. 9, 2018. The entire disclosures of which are incorporated herein by reference in their entirety.

### STATEMENT REGARDING FEDERALLY FUNDED RESEARCH

Not applicable.

### BACKGROUND

An autograft can refer to tissue transplanted from one part of an individual's body (e.g., a “donor site”) to another part (e.g., a “recipient site” or a “wound site”). Autografts can be used, for example, to replace missing skin and other tissue and/or to accelerate healing resulting from trauma, wounds, burns, surgery, and birth defects. Generally, grafting procedures can be limited by the amount of tissue that can be removed from the donor site without causing excessive adverse effects. More specifically, availability of tissue for autografting can be limited by a total area of tissue needed, healing behavior of the donor site, similarity of the donor and recipient sites, aesthetic considerations, and/or other characteristics of candidate donor and/or recipient sites.

A sheet graft is one type of autograft and refers to a piece of tissue that is removed, or harvested, from an undamaged donor site. For example, a sheet graft may be obtained using an instrument structured to gently shave a piece of tissue from the skin at the donor site. The size of the donor skin piece used for the graft may be about the same size as the damaged recipient site, slightly larger (e.g., to account for potential shrinkage of the graft tissue after harvesting), or slightly smaller (e.g., and then meshed and expanded to increase coverage area and facilitate drainage once grafted). Once harvested, the sheet graft can be applied over the recipient site wound, stapled or otherwise fastened in place, and allowed to heal.

Sheet grafts can be full-thickness or split-thickness. For example, a conventional split-thickness graft can be formed by harvesting a sheet of epidermis and upper dermal tissue from a donor site, whereas full-thickness skin grafts can be formed using sheets of tissue that include the epidermis layer and the entire dermal component. The type of sheet graft used can affect healing at both the donor site and the recipient site.

For example, in conventional split-thickness grafts, the skin tissue may grow back at the donor site in a process similar to that of healing a second-degree burn. Split-thickness grafts may thus be preferable to full-thickness grafts because of the higher availability of donor sites. However, skin tissue removed from the donor site for a split-thickness skin autograft generally includes only a thin epithelial layer, which can lack certain elements of the dermis that would improve function, structural stability, and normal appearance at the recipient site once healed.

In conventional full-thickness grafts, more characteristics of normal skin, such as color, texture, and thickness, can be maintained at the recipient site following the grafting procedure (i.e., because the dermal component can be preserved in such grafts). For example, full-thickness grafts can contain a greater collagen content, dermal vascular plexus, and epithelial appendages as compared to split-thickness grafts. Full-thickness grafts may also undergo less contraction while healing. These properties can be important on more visible skin areas, such as the face and hands. Additionally, hair can be more likely to grow from full-thickness grafts than from split-thickness grafts, and sweat glands and sebaceous glands can be more likely to regenerate in full-thickness grafts than in split-thickness grafts, taking on the sweating characteristics of the recipient site.

While full-thickness grafts can provide improved tissue quality at the recipient site, the donor site is completely sacrificed and must be closed by primary closure. Additionally, full-thickness grafts require more precise conditions for survival because of the greater amount of tissue requiring revascularization. As such, conventional full-thickness skin grafts are generally limited to relatively small, uncontaminated, well-vascularized wounds, and may not be appropriate for as many types of graft procedures as split-thickness grafts.

In light of the above, it may be desirable to provide systems and methods for tissue harvesting and grafting that provide efficient graft tissue with minimal donor site scarring while also properly replicating normal tissue microanatomy at the recipient site. Additionally, it is desirable for such systems and methods to be scalable for use at recipient sites of various sizes and shapes.

### SUMMARY

The systems and methods of the present disclosure overcome the above and other drawbacks by providing fractional tissue grafts, in the form of full-thickness micro tissue columns, that maintain a desired orientation of the individual tissue columns, such as a substantially vertical, epidermal-dermal orientation. The systems and methods of the present disclosure further optimize grafting procedures by analyzing a recipient site, selecting an optimal donor site based on characteristics of both sites, including graft depth, and using a hydraulic transfer tool to harvest and transfer the micro tissue columns.

In accordance with one aspect of the disclosure, a system for assembling a plurality of micro tissue grafts into a custom graft includes a pick and place unit, a transfer tool, and a control unit. The pick and place unit includes a moveable arm and the transfer tool is coupled to a distal end of the arm. The control unit is configured to control the moveable arm to move the transfer tool to a donor site and a graft construction site. The control unit is further configured to control the transfer tool to harvest the micro tissue grafts from the donor site and arrange the micro tissue grafts into a supportive matrix to form the custom graft.

In accordance with another aspect of the disclosure, a method for assembling a plurality of micro tissue grafts is provided. The method includes analyzing a recipient site by imaging the recipient site and creating a wound profile, and selecting a donor site based on the wound profile. The method also includes harvesting the plurality of micro tissue grafts from the donor site, arranging the plurality of micro tissue grafts in a desired orientation within a supportive material to create a custom graft, and applying the custom graft to the recipient site.

In accordance with yet another aspect of the disclosure, a method for assembling a plurality of micro tissue grafts using a system including an arm, a transfer tool, and a sensor is provided. The method includes creating a wound profile by positioning the sensor over a recipient site and imaging the recipient site, positioning the sensor over a donor site to map the plurality of micro tissue grafts within the donor site based on the wound profile, and extracting the plurality of micro tissue grafts from the donor site into the transfer tool using hydraulic pressure. The method also includes extruding the plurality of micro tissue grafts from within the transfer tool into a supportive material to create a custom graft, and applying the custom graft to the recipient site.

In accordance with a further aspect of the disclosure, a method for harvesting a micro tissue graft from a donor site to into a supportive material using a hydroextractor is provided. The method includes contacting a surface of the donor site with the hydroextractor so that a lower surface of a hollowing cutting tool and a lower surface of an internal pin of the hydroextractor contact the surface of the donor site and delivering a liquid between the pin and the hollow cutting tool so that the liquid travels to the surface of the donor site creating a hydraulic coupling between the hydroextractor and the surface. The method also includes moving the hollow cutting tool downward into the tissue while the pin remains flush with the surface to harvest the micro tissue graft within a tissue space of the hollow cutting tool, where the liquid seals the micro tissue graft within the tissue space, and retracting the hydroextractor away from the surface of the donor site to extrude the micro tissue graft from the donor site. The method further includes contacting a surface of the supportive material with the hydroextractor so that the lower surface of the hollow cutting tool contacts the surface of the supporting material, lowering the hydroextractor into the supportive matrix until the pin is level with the surface of the supportive matrix, and retracting the hollow cutting tool from the supportive matrix so that the micro tissue column remains within the supportive matrix.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic diagram of a system for harvesting, organizing, and assembling tissue grafts.

FIG. 2 is a diagram illustrating a portion of donor site tissue and a portion of a custom graft including micro tissue columns harvested from the donor site tissue.

FIG. 3 is a schematic diagram of a hydroextractor of a transfer tool of the system of FIG. 1.

FIG. 4 is a diagram illustrating a method for extracting and extruding micro tissue columns using the hydroextractor of FIG. 3.

FIG. 5 is a flow diagram illustrating a method for organizing and assembling tissue grafts.

### DETAILED DESCRIPTION

The disclosure provides systems and methods for planning, extruding, organizing, assembling, and placing tissue grafts. More specifically, the present systems and methods enable assembling custom micro tissue grafts, in the form of biological micro tissue columns, in a way that maintains a desired orientation of the individual tissue columns. The present system can maintain a precise shape and depth of the custom graft through graft harvesting, transfer, and placement processes from a donor site to a recipient site. Furthermore, the system uses a hydraulic coupling to extract consistent micro tissue grafts with minimal disruption or dislocation of tissue layers within the micro tissue grafts.

For example, full-thickness skin tissue can be harvested from a donor site in the form of small columns (e.g., several hundred micrometers in diameter) without causing scarring at the donor site. Because skin is naturally polarized in architecture, engrafting micro tissue columns as an array having a proper epidermal-dermal orientation into the wound bed of a recipient site can further improve healing by accelerating re-epithelialization processing, recapitulating normal dermal architecture, and reducing scarring. As such, the methods and systems disclosed herein facilitate analyzing a recipient site, selecting a proper donor site, extracting micro tissue columns, orienting micro tissue columns and enabling their assembly into a custom three-dimensional, full-thickness graft, and applying the custom graft to a recipient site. The present systems and methods also provide a practical, scalable solution for using large numbers of micro tissue columns to improve healing wounds of various sizes and shapes.

The discussion below generally refers to grafts or autografts. It should be noted that, in some applications, the systems and methods described herein may apply to autografts or, alternatively, allografts. Furthermore, while the term micro tissue columns, or MTCs, is used herein, it should be noted that this term may be interchangeable with micro tissue grafts or micrografts. Also, when the subject tissue is skin, MTCs may be referred to as micro skin tissue columns (MSTCs).

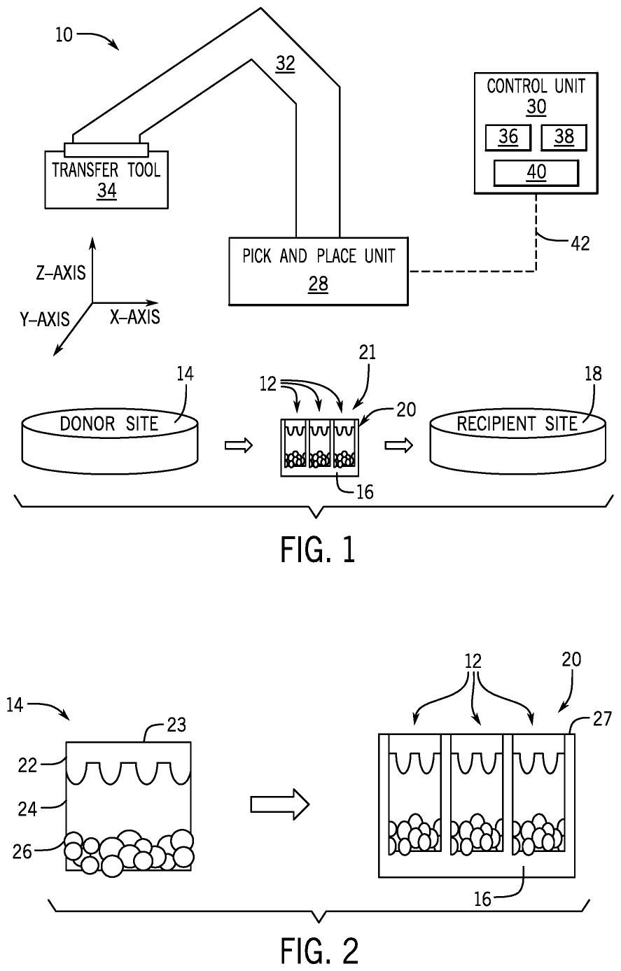

FIG. 1 illustrates a system 10, according to some aspects of the present disclosure, configured to create custom grafts with precise shape and depth. Generally, the system 10 is a hydraulic transfer system configured to “pick and place” full-thickness MTCs 12 from a donor site 14 into a supportive matrix 16 (e.g., a biocompatible substrate) for placement at a recipient site 18.

In other words, the system 10 is configured to extract MTCs 12 from the donor site 14 and extrude the MTCs 12 in a desired orientation (e.g., matching an epidermal-dermal polarity of normal skin) and, optionally, the same relative spatial organization as the donor site 14 into a supportive matrix 16 to create a custom graft 20. The system 10 organizes, orients, positions, and assembles harvested MTCs 12 onto or into the matrix 16 so that the custom graft 20 maintains its precise shape and depth throughout transfer and placement to the recipient site 18. By way of example, FIG. 2 illustrates a portion of a donor site 14 and a portion of a custom graft 20. As shown in FIG. 2, the MTCs 12 harvested from the donor site 14 into the graft 20 can be full-thickness grafts, including the epidermal layer 22 (e.g., adjacent a surface 23 of the donor site tissue) as well as a dermal layer 24 and, optionally, a portion of a dermal/fatty layer boundary 26.

Referring back to FIG. 1, the system 10 can include a robotic pick and place unit 28 coupled to a control unit 30. The pick and place unit 28 can include a movable arm 32 and a hydraulic transfer tool 34 positioned at a distal end of the arm 32. The hydraulic transfer tool 34 can be mechanically and/or electrically coupled to the movable arm 32. Furthermore, the transfer tool 34 can be permanently or removably coupled to the moveable arm 32. For example, in one aspect, the hydraulic transfer tool 34 can be permanently or removably coupled to a strain gauge (not shown) at the distal end of the movable arm 32.

The control unit 30 can be a computer, tablet, or other suitable computing device including a processor 36, memory 38, and a user interface 40. The processor 36 can be configured to execute a program (e.g., stored in the memory 38) to control the pick and place unit 28 in accordance with instructions stored in the memory 38 and/or received by the user interface 40. For example, the control unit 30 can control movement of the arm 32 (e.g., along X, Y, and/or Z axes) and movement (e.g., along the Z axis) and operation of the transfer tool 34. In some aspects, the control unit 30 can be coupled to the pick and place unit 28 via a wired connection 42, such as a USB cable or another suitable wired connection, or a wireless connection (not shown). In some aspects, the control unit 30 can be an internal specialized computer embedded within the pick and place unit 28. Additionally, in some aspects, the control unit 30 can be connected to the hydraulic transfer tool 34 via the pick and place unit 28 and the movable arm 32 or can be separately connected to the hydraulic transfer tool 34.

Generally, the pick and place unit 28 can function as a transfer station for MTCs 12. A transfer plane can consist of the donor site 14 and the recipient site 18 located adjacent each other (e.g., on a benchtop for testing or in an operating room during use with a patient). Alternatively, the transfer plane can consist of the donor site 14 and a graft construction site 21 (e.g., consisting of the matrix 16 for receiving MTCs 12). For example, the graft construction site 21 may be at the recipient site 18 or may be remote from the recipient site 18.

In some aspects, the pick and place unit 28 can be configured to move the transfer tool 34, via the arm 32, along two axes (i.e., along the X-axis and Y-axis) +/− about four inches, about eight inches, or other suitable distances to enable MTC transfer from the donor site 14 to the recipient site 18 (or to the graft construction site 21). The pick and place unit 28 can further be configured to move the transfer tool 34 along a third axis (i.e., the Z-axis) in order to cut into the donor site 14 for a desired depth, such as to cut into the dermal and epidermal layers 22, 24 of the donor site 14, and to cut into the graft construction site 21 for a desired depth, such as to place the MTCs 12 into the matrix material 16. Furthermore, an operator can program the system 10 for different rates and speeds of movement along the X, Y, and Z axes to achieve an accurate MTC transfer as part of a calibration process. For example, in one application, the system 10 can be configured to transfer one MTC 12 per second from the donor site 14 to the graft construction site 21. In another application, the system 10 can be configured to transfer two MTCs 12 per second from the donor site 14 to the graft construction site 21. In yet other applications, other transfer rates may be configured. Also, in some applications, the pick and place unit 28 can weigh less than about ten pounds.

In some aspects, the transfer tool 34 can be sized and configured for locating, extracting, extruding, orienting, and/or placing MTCs 12. Generally, the transfer tool 34 can at least include a hollow cutting tool assembly with X-, Y-, and/or Z-axis motion capabilities for extracting, extruding, and placing MTCs 12. For example, in some aspects, the transfer tool 34 is capable of independent Z-axis motion, while X- and Y-axis motion is completed by the moveable arm 32. In some aspects, however, X-, Y-, and Z-axis motion is controlled by the moveable arm 32. Additionally, all motion can be controlled by the control unit 30. Alternatively, a separate controller (not shown) may be configured to control Z-axis motion of the transfer tool 34.

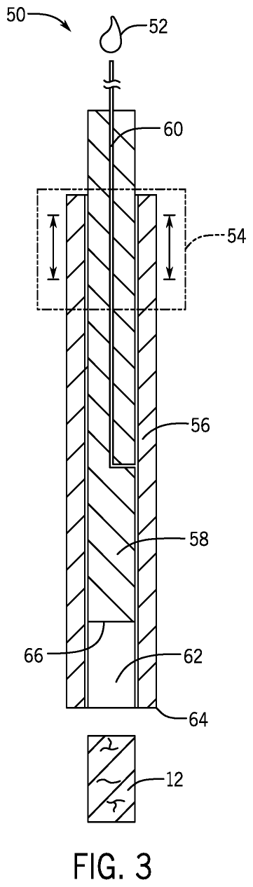

The transfer tool 34 can include one or more of the following: a hydroextractor 50 (as shown in FIG. 3), a liquid source 52, one or more linear actuators 54, one or more position sensors, one or more image sensors, and a digital camera. Each of these components can be controlled by and/or in communication with the control unit 30 and/or a separate controller. For example, the hydroextractor 50, along with the liquid source 52, the linear actuators 54, and/or the position sensors may be controlled to harvest and extrude MTCs 12, and the image sensors and/or the digital camera may be used to analyze and select donor and recipient sites. Additionally, one or more of these components may be removable from the transfer tool 34 (e.g., for custom use or for disposability).

As shown in FIG. 3, the hydroextractor 50 of the transfer tool 34 can include a hollow cutting tool 56, a pin 58, and a liquid delivery system 60. In some aspects, the hydroextractor 50 can be a replaceable component of the transfer tool 34. For example, the hydroextractor 50 can be a separate sterile kit configured to be clipped into or otherwise coupled to the transfer tool 34. In one aspect, the hydroextractor 50 can clip into a Z-axis solenoid of the transfer tool 34 (or of the moveable arm 32). In some applications, the hydroextractor 50 can be comprised of metal, plastic, or a combination of materials.

In some non-limiting examples, the hollow cutting tool 56 can be sharpened at its inner edges (e.g., angled upward from its inner edge to its outer edge, or tapered to form a sharpened top at a lower edge 64 of the cutting tool 56), and can be rounded or square in shape (or another suitable shape), with the pin 58 positioned inside the tool 56. In some non-limiting examples, the pin 58 may be slidably received within the hollow cutting tool 56. In one application, the hollow cutting tool 56 can include an outer diameter of approximately 0.785 millimeters (about 1/32 of an inch).

The linear actuators 54 can control Z-axis movement of the entire hydroextractor 50 and can also control Z-axis movement of the hollow cutting tool 56 independently of the other components. For example, the linear actuators 54 can be configured to move the hollow cutting tool 56 relative to the pin 58, creating a tissue space 62 within a distal end of the hollow cutting tool 56, as shown in FIG. 3. During use, an MTC can be extracted into the tissue space 62 and maintained within the tissue space 62 until extruded at the graft construction site 21 or the recipient site 18.

Furthermore, the liquid delivery system 60 can include or be in communication with the liquid source 52 and be configured to deliver liquid within the hollow cutting tool 56. The liquid source 52 can be, for example, a riding vial, injectable reservoir, or other compartment configured to hold a volume of liquid (such as sterile saline solution), or can be an attachment point for an externally connected source of such liquid (such as an IV bag, a syringe, a bottle, or other source). The liquid delivery system 60 can be a passive or active system that delivers a micro-controlled column of fluid from the liquid source 52 to a delivery point between the pin 58 and the cutting tool 56, allowing the fluid to surround the pin 58 within the cutting tool 56 and travel downward into the tissue space 62. By way of example, passive systems may be gravity-fed systems, while active systems may be pump-controlled systems. In some aspects, the liquid delivery system 60 can deliver fluid at a metered rate, such as one drop per hour, or another rate.

The liquid delivery system 60 can provide a micro-controlled adjusted column of fluid that is always in contact with an MTC 12 from the time it is extracted from the donor site 14 into the cutting tool 56, transferred hydraulically, and extruded into the supportive matrix 16 (e.g., at the graft construction site 21). As further described below, liquid delivery within the hydroextractor 50 creates a “liquid piston” inside the cutting tool 56 that continuously contacts an extracted MTC 12 within the tissue space 62 and acts as a sealing mechanism between the MTC 12, the cutting tool 56, and the pin 58. Furthermore, skin is composed of approximately 64% water and the constant bathing of MTCs 12 via the liquid delivery system 60 can reduce cellular damage from dehydration during the transfer process.

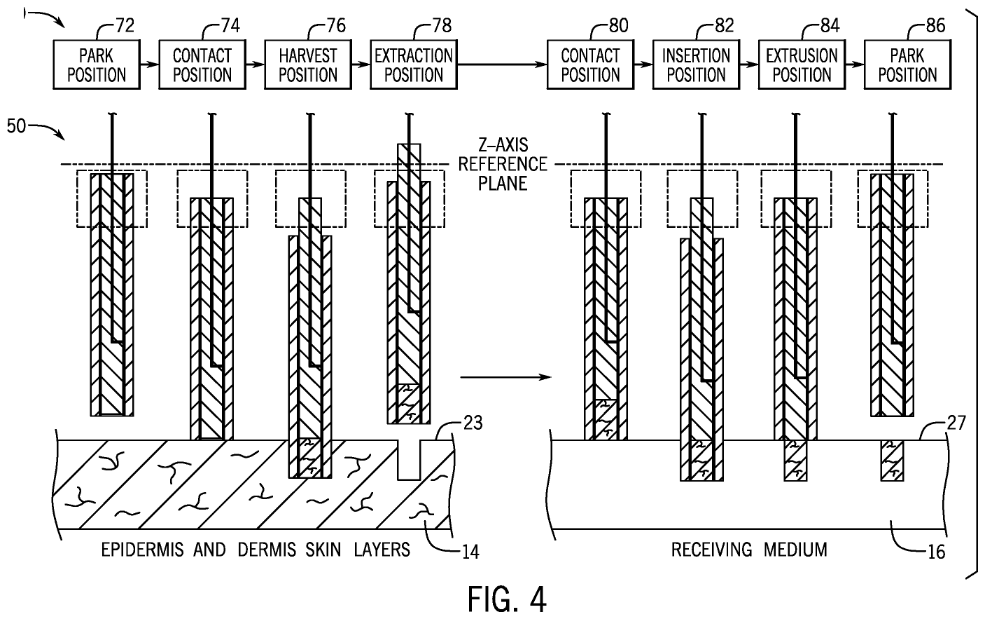

Accordingly, the hydroextractor 50 can individually extract MTCs 12 using the hollow cutting tool 56 with the pin 58, along with the hydraulic coupling created by the liquid delivery system 60. For example, FIG. 4 illustrates a pick and place transfer method 70, according to some embodiments, using the hydroextractor 50. Generally, as shown in FIG. 4, the hydroextractor 50 is initially placed in a “park position” above a desired location at the donor site 14 at step 72. At step 74, the hydroextractor 50 is moved along the Z-axis into a “contact position” where it contacts the surface 23 of the donor site 14. At step 76, the hydroextractor is moved into a “harvest position” where the cutting tool 56 is inserted a depth into the donor site 14 to harvest a MTC 12 within the tissue space 62. At step 78, the hydroextractor 50, including the MTC 12, is moved into an “extraction position” above the donor site 14. At step 80, the hydroextractor 50 and MTC 12 are transferred to a desired location of the graft construction site 21 and placed into a contact position where it contacts a surface of the supportive matrix 16. At step 82, the hydroextractor 50 is moved into an “insertion position” where the cutting tool 56 and MTC 12 are inserted a depth into the matrix 16. At step 84, the hydroextractor 50 is moved into an “extrusion position” where the cutting tool 56 is retracted from the matrix 16 so that the MTC 12 is retained within the matrix 16. Finally, at step 86, the hydroextractor 50 is moved into a park position above the graft construction site 21 and ready to be moved back to a new park position at the donor site 14.

More specifically, at step 72, the hydroextractor 50 is initially placed in a park position above a desired location at the donor site 14. The desired location can be determined by the control unit 30, as further described below, and the control unit 30 can control X-Y movement of the moveable arm 32 to move the hydroextractor 50 above the desired location. As shown in FIG. 4, in the park position, the hydroextractor 50 hovers a distance above the surface 23 of the donor site 14.

At step 74, the control unit 30 can control Z-movement of the hydroextractor 50 (e.g., via the linear actuators 54) to move the hydroextractor 50 into a contact position, where it contacts the surface 23 of the donor site 14. More specifically, as shown in FIG. 4, a lower surface (or lower edge) 64 of the hollow cutting tool 56 and a lower surface 66 of the pin 58 can be flush with the donor site surface 23 while in the contact position. The control unit 30 can control Z-movement of the hydroextractor 50 downward until these lower surfaces 64, 66 contact the donor site surface 23 and, in some aspects, the transfer tool 34 (or the hydroextractor 50) can include one or more position sensors (not shown) to provide feedback to the control unit 30 indicating when the lower surfaces contact 64, 66 contact the donor site surface 23. For example, transducers, electrostatic sensors, or other pressure or proximity measurement tools may be used to provide feedback to the control unit 30 regarding Z-axis positioning of the hydroextractor 50 relative to the tissue surface 23. In one example, as noted above, the transfer tool 34 can include one or more pressure sensors, such as one or more load cells configured to provide feedback the control unit 30 so that the control unit 30 may determine when the hydroextractor 50 contacts the surface 23 of the donor site 14.

Additionally, during and following step 74 (and, in some aspects, starting at the park position step 72), the liquid delivery system 60 is operating to deliver liquid between the pin 58 and the hollow cutting tool 56. The liquid delivery wets the surface 23 of the tissue, which creates a hydraulic coupling between the hydroextractor 50 and the donor site tissue.

At step 76, the hydroextractor is moved into a harvest position, where the control unit 30 controls one of the linear actuators 54 to move the cutting tool 56 downward relative to the pin 58. That is, the lower surface 64 of the cutting tool 56 extends past the lower surface 66 of the pin 58, while the lower surface 66 of the pin 58 remains in contact with the surface 23 of the donor site 14. As a result, the cutting tool 56 is inserted a depth into the donor site 14 while the pin 58 remains flush with the tissue surface 23, allowing an MTC 12 to occupy the tissue space 62 within the cutting tool 56. The penetration depth of the hollow cutting tool 56 into the donor site 14 (i.e., the Z-axis movement of the hollow cutting tool 56 from the contact position to the harvest position), corresponding to the height of the harvested MTC 12, can be preprogrammed through the control unit 30. In some aspects, a single hydroextractor 50 can be configured to extend to variable tissue depths to achieve different MTC heights. In other aspects, different hydroextractors 50 may be used to achieve different MTC heights. For example, the transfer tool 34 can be configured to connect to different hydroextractors 50 (e.g., for replacement or sterilization purposes and/or to achieve different MTC heights).

As noted above, the liquid delivery system 60 operates to deliver liquid between the pin 58 and the hollow cutting tool 56 (e.g., in the radial, or lateral space between the outer surface of the pin 58 and the inner surface of the cutting tool 56). As a hydraulic system, the liquid acts as a solid mass creating a hydraulic coupling of the cutting tool 56, the pin 58, and the donor site tissue. Furthermore, the hydraulic coupling generally acts as liquid piston that moves with the hollow cutting tool 56 as it is lowered into the donor site tissue. More specifically, the system 10 can put pressure on the cutting tool 56 (e.g., about 0.4 pounds per square inch), which translates into fluidic pressure. When the hydraulic pressure reaches a critical point, the solid mass breaks through the tissue, displacing the MTC 12 into the tissue space 62 of the cutting tool with minimal disruption to the epidermis and dermis. The hydraulic coupling provides a liquid seal that minimizes friction as the donor site tissue is pierced and the cutting tool 56 moves downward, and bathes the MTC 12 in fluid to help minimize tissue lysing and disturbance of tissue layers. Furthermore, as noted above, the cutting tool 56 can have sharp edges, which can help minimize lysing of cells during this process. At this point, the solid mass created by the hydraulic coupling also includes the MTC 12 to be extracted.

At step 78, hydroextractor 50, including the MTC 12, is moved into an extraction position, where the control unit 30 withdraws the hydroextractor 50 upward above the donor site 14 (e.g., via the linear actuators 54). In other words, the applied pressure can be reversed so that the hydraulics move the solid mass upward, lifting the hydroextractor 50 and MTC 12 just above the original tissue surface 23, while the donor site tissue space previously occupied by the MTC 12 is filled with fluid from the liquid delivery system 60. Because the MTC 12 is sealed within the tissue space 62 of the cutting tool 56, due to the hydraulic coupling, it is also extracted.

More specifically, the hydraulic coupling, along with rapid movement of the hydroextractor throughout the harvest position and extraction position steps 76, 78, creates a vacuum to seal the MTC 12 within the tissue space 62. The rapid movement of the hydroextractor 50, while the liquid delivery system 60 is operating, can create a momentary pressure sufficient enough to seal the MTC 12 in the hollow cutting tool 56. As such, no other sealing mechanisms are necessary to maintain the MTC 12 within the tissue space 62. Furthermore, due to the hydraulic coupling, the MTC 12 is never physically compressed during this process. As a result, the hydroextractor 50 can produce consistent, full, undisturbed MTCs 12, e.g., compared to other techniques such as punching tools or arrays that may provide inconsistent, stringy columns.

At step 80, the hydroextractor 50 and MTC 12 are transferred to a desired location at the graft construction site 21 and placed into a contact position along a surface 27 of the supportive matrix 16. More specifically, the control unit 30 controls X-Y movement of the moveable arm 32 to move the hydroextractor 50 to the desired location at the graft construction site 21, and controls the Z-movement of the hydroextractor 50 (e.g., via the linear actuators 54) to move the hydroextractor 50 downward until it contacts the surface 27 of the matrix 16. For example, the hydroextractor 50 is in the contact position when the lower surface 64 of the hollow cutting tool 56 contacts the surface 27 of the matrix 16. The control unit 30 can determine and locate the desired location, as described below, and can determine when the hydroextractor 50 is at the necessary depth in the contact position via sensor feedback, as described above.

At step 82, the hydroextractor 50 is moved into an insertion position where the cutting tool 56 and MTC 12 are inserted a depth into the matrix 16. For example, the applied pressure on the solid mass (including the hydraulically coupled MTC 12) can be reversed so that the hydraulics move the MTC 12 downward, penetrating the supportive matrix 16 to a programmed Z-axis depth. More specifically, the cutting tool 56 and MTC 12 can be inserted the same depth into the matrix 16 during the insertion position as they were inserted into the donor site tissue during the harvest position at step 76, so that the lower surface 66 of the pin 58 is approximately level with the surface 27 of the matrix 16.

Because the MTC 12 was sealed in the tissue space 62 during harvest and extraction, it maintains its same vertical orientation as it had while at the donor site 14. Furthermore, the MTC 12 maintains its same vertical orientation once inserted into the matrix 16. In some embodiments, the supportive matrix 16 may be liquid or a low density fibrous collagen material, or another suitable matrix type that may be easily displaced during insertion of the hollow cutting tool 56 and the MTC 12, as further described below.

At step 84, the hydroextractor 50 is moved into an extrusion position where the cutting tool 56 is retracted from the matrix 16 so that it is again flush with the pin 58 and the MTC 12 is retained within the matrix 16. For example, the MTC 12 can be “extruded” into the fibrous collagen matrix 16 by lowering the applied pressure to the surrounding atmospheric pressure on the enclosed liquid, the cutting tool 56, and the pin 58 and, thus, releasing the hydro-coupling. By releasing the hydraulic coupling effect, the cutting tool 56 can then be lifted upward with the MTC 12 remaining properly aligned in the fibrous collagen of the matrix 16. The lower surface 66 of the pin 58 can act as a stop to prevent the MTC 12 from retracting the hollow cutting tool 56 as it is withdrawn from the matrix 16. Accordingly, at the end of the extrusion step 84, the hydroextractor 50 is flush with the matrix surface 27 while the MTC 12 is maintained within the matrix 16, in the same vertical orientation as it was in the donor site 14. For example, the supportive matrix 16 can help maintain the donor site structural memory of both the epidermis and dermis donor-site levels of the MTC 12.

At step 86, the entire hydroextractor 50 is moved upward into a park position above the graft construction site 21 and ready to be moved back to a new park position at a new desired location at the donor site 14. The control unit 30 can then repeat the above process steps 72-86 to harvest and extrude additional MTCs 12 to prepare a custom three-dimensional graft 20 having a desired size and shape as well as MTCs 12 are that are vertically, horizontally, and rotationally oriented relative to each other in a desired manner.

More specifically, the system 10 can extrude the MTCs 12 in proper alignment in the supportive matrix 16 regarding both upright (horizontal) positioning and positioning relative to their point of origin at the donor site 14. For example, MTCs 12 can be vertically and rotationally oriented in a similar manner as they were while in their original positions at the donor site 14. Furthermore, the MTCs 12 can be horizontally oriented relative to each other in a similar or different manner as they were while in their original positions at the donor site 14. For example, in some aspects, the MTCs 12 can be closer together or further spaced apart compared to their relative original positions within the donor site 14.

Furthermore, the system 10 can create the custom graft 20 to have a specific depth, length, width, and shape. In some aspects, the shape may be a square or rectangle with a uniformly defined length and width; however, the graft 20 may take other custom shapes in some aspects. In one application, the system 10 can be configured to achieve 900 MTCs in a 1-inch by 1-inch graft 20 of properly aligned, matrixed MTCs 12. However, in other applications, the system 10 can be configured for other MTC amounts and graft sizes. Additionally, while the above process 70 is described with respect to extruding MTCs 12 into a custom graft 20 at the graft construction site 21, in some applications, the process 70 may instead extrude individual MTCs 12 directly into a recipient site 18.

In light of the above, there are three types of independent controls of the transfer tool 34, as controlled by the control unit 30: (1) the fluid flow throughout the transfer process; (2) the motion of the hydroextractor 50 in the X, Y and Z directions; and (3) the motion of the hollow cutting tool 56 along the Z-axis for extracting and extruding individual MTCs 12. According to some aspects, the single-element concept of the transfer tool 34 stands in contrast to existing needle arrays used for MTC harvesting. More specifically, as described above, the transfer tool 34 and the hydroextractor 50 harvest one MTC 12 at a time, allowing for customized grafts with different relative horizontal MTC orientations, sizes, and shapes. In contrast, needle arrays create multiple skin piercings simultaneously. Such arrays have needles that are horizontally oriented relative to one another and, thus, only allow a single relative orientation of MTCs. Furthermore, needle arrays, by already being a set shape and size, can limit in the type of resulting graft shapes and sizes.

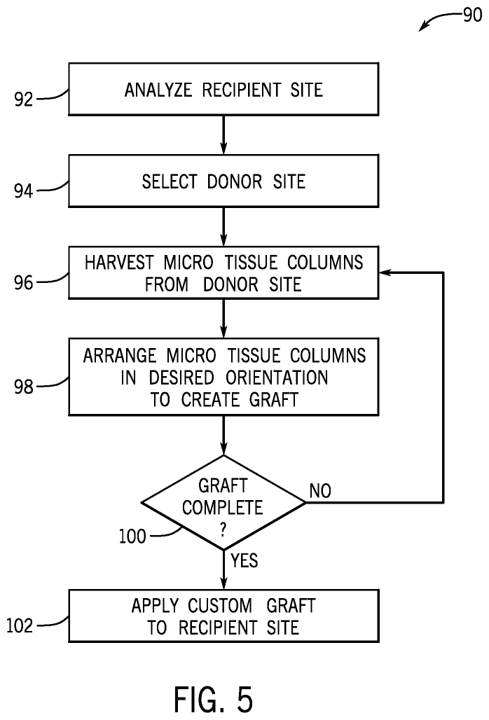

Referring now to FIG. 5, a method 90 for assembling MTCs in accordance with the present disclosure is provided. Generally, in accordance with this method 90, the system 10 (or another suitable system) can digitize the shape and depth of the recipient site 18 for preparation of a custom graft 20 and program MTC location, harvesting, organization, orientation, transfer, and placement as an autograft skin construct from the donor site 14 to the recipient site 18. More specifically, as shown in FIG. 5, the recipient site 18 can be analyzed at step 92, a donor site 14 is selected at step 94, and MTCs 12 are harvested from the donor site 14 at step 96. At step 98, MTCs 12 are arranged in a desired orientation (e.g., matching an epidermal-dermal polarity of normal skin) to create a custom graft 20. Steps 96 and 98 may then be repeated to complete the custom graft 20, as determined at step 100. And at step 102, once the custom graft 20 is completed, the custom graft 20 is applied to the recipient site 18. Alternatively, the custom graft 20 is stored in a sterile environment for a time period before being applied to the recipient site 18.

With respect to step 92, prior to any tissue harvesting, the recipient site 18 can be imaged. For example, a digitized picture can be taken of the recipient site 18 via one or more image sensors of the system 10, such as a standalone digital camera or a digital camera of the transfer tool 34. In some aspects, the digital camera can be in communication with the control unit 30, providing the digitized image as input to the control unit 30. From the digitized picture, the system 10 can be programmed to create a software profile of the full-thickness graft to be harvested in the form of a precisely-described autograft (e.g., a “MTC Wound Profile’) stored in memory (e.g., memory 38) as X, Y and Z coordinates. The MTC Wound Profile can define the shape and depth of the autograft 20 and includes the location, orientation and placement for each potential MTC 12 to be harvested.

With respect to step 94, once the system 10 has established an MTC Wound Profile, the system 10 can analyze one or more potential donor sites 14 to determine an optimal donor site 14 for MTC harvesting, including available depth of the skin layers. More specifically, the system 10 can be placed over the surface 23 of a potential donor site 14 (such as an autograft donor site), and can include sensors to locate the surface 23 of the donor site 14 based on X, Y, and Z coordinates. For example, the image sensors of the transfer tool 34 can be used to acquire data (such as digital images or three-dimensional image data) of the potential donor site 14 and input such data to the control unit 30. In some aspects, the image sensors can be integrated into the transfer tool 34 and electronically connected to the control unit 30 (e.g., via a transducer connected at the strain gauge of the moveable arm 32) for digital readings and analysis by the control unit 30. Using the information gained and stored from the MTC Wound Profile in step 92, the system 10 can then be programmed to generate a precise, detailed MTC harvesting map at the donor site 14 (e.g., an “MTC Skin Construct Plan”).

For example, commercial digitization technology can be used to precisely measure the depth of the wound at the recipient site 18, which informs the Z coordinate of the MTC Skin Construct Plan. In addition to the epidermis and dermis skin layers 22, 24 to be harvested, the system 10 can also consider and control harvesting more or less of the fat skin layer according to the depth of the wound. As noted above with respect to FIG. 2, each MTC 12 can be a full-thickness graft, including the epidermal layer 22 as well as a dermal layer 24 and, optionally, a portion of a dermal/fatty layer boundary 26. In general, it can be preferable to harvest MTCs 12 with epidermal tissue and dermal tissue, while avoiding a significant amount of subcutaneous tissue or muscle tissue (though, in some applications, MTCs 12 can include subcutaneous tissue and/or muscle tissue).

For example, in some applications, each MTC 12 can be about 3 mm in height, which can correspond to a total depth of a typical skin layer (e.g., including both epidermal and dermal layers 22, 24, where the dermal layer includes hair follicles and sweat or sebaceous glands). A different depth may be used, such as between about 2 mm and about 4 mm, based on the particular skin or tissue characteristics of the donor site 14 and requirements of the recipient site 18. Additionally, some donor sites 14 will provide MTCs 12 including stem cells throughout the dermal tissue (e.g., stem cells associated with hair follicles and sweat glands and/or stem cells in a lower portion of the dermal layer 24, for example, near a dermal/fatty layer boundary 26). Accordingly, in addition to selecting a particular tissue depth, the system 10 can be further selective as to harvesting of particular MTCs 12, including opting to select or deselect hair follicles, or opting to select pieces of donor site tissue not to be harvested, such as skin covered by a mole. In some aspects, these desired or disfavored features can be determined via the acquired image data.

Once the MTC Skin Construct Plan is completed, the system 10 can be moved to a new potential donor site 14 and a new MTC Skin Construct Plan can be determined. This process can be repeated and a new MTC Skin Construct Plan can be determined for each potential donor site 14 prior to the cutting of any tissue. Finally, the MTC Skin Construct Plans can be compared and a final donor site 14 (or donor sites 14) can be selected.

Accordingly, at step 96, each MTC Skin Construct Plan provides a software representation of the MTCs 12 to be harvested in an organized manner from a donor site 14. And the MTCs 12 will be transferred by precise location, orientation and placement in any shape and depth as informed by the MTC Wound Profile, as determined at step 92. Steps 92 and 94 thus provide the ability to procedurally organize the assembly of a precise, custom graft 20 in silico prior to the cutting of any tissue.

Referring now to step 96, the MTCs 12 can be harvested from the selected donor site 14. More specifically, MTCs 12 can be formed by removing elongated, substantially cylindrical portions of tissue from the donor site 14, thus leaving holes therein. Generally, the diameter or width of an MTC 12 can correspond to an internal diameter of the cutting tool 56 of the transfer tool 34. For example, in some embodiments, a diameter or width of an MTC 12 can be less than about 2 millimeters (mm) or less than about 1 mm. In some embodiments, the diameter or width can be less than about 0.5 mm, less than about 0.3 mm, or about 0.2 mm. In further embodiments, the diameter or width can be between about 0.8 mm and 0.3 mm. In other embodiments, the diameter or width can be between about 0.7 mm and 0.2 mm.

Generally, the MTCs 12 can be harvested from the donor site 14 in a way that minimizes or prevents scarring at the donor site. For example, a size of a donor site hole created by a respective MTC 12 can be selected so that the minor damage created heals rapidly and/or without scarring. More specifically, each donor site hole can be small enough to heal quickly by regeneration, that is, by replacement of the harvested tissue volume with new skin tissue that is normal in both structure and function, without or with minimal scarring. Additionally, the size of the donor site holes created by the MTCs 12 can be selected based on creating portions of tissue that can be small enough to promote viability when transplanted or placed in a growth medium, and large enough to form a sufficient amount of graft tissue and/or to capture tissue structures that may be present in the donor tissue.

Generally, the fraction of surface tissue removed from the donor site 14 (which can correspond to a fractional surface area of the donor site 14 occupied by the holes) can correspond to the relative harvesting arrangement determined by the control unit 30. In some embodiments, the fraction of surface tissue removed from the donor site can be less than about 70%, less than or equal to about 50%, or more preferably between about 10% and about 30%. The fraction of tissue removed can be sufficiently large to provide enough harvested MTCs 12 to form an appropriately sized graft 20, but small enough to facilitate rapid healing at the donor site 14 based on growth from the remaining undamaged tissue. Other fractions of tissue can be removed from a donor site 14 depending on factors such as, for example, the particular characteristics of the donor site 14, the size of the graft 20 needed, and the overall amount of donor site tissue available.

According to some embodiments, the MTCs 12 can be harvested at step 96 using the above-described transfer tool 34 of the system 10 and the method 70 described above with respect to FIG. 3. For example, the transfer tool 34 can be attached to a strain gauge connected to a transducer that measures the force profile of the transfer tool 34 during operation. The MTC Skin Construct Plan can include a calculated range for an acceptable force profile during operation of the system 10. The system 10 can control and maintain the proper force profile for the transfer tool 34 during all stages of operation of the transfer tool 34 via this sensor mechanism and software (e.g., one or more programs saved in memory 38 and executed by the processor 36).

As described above, during extraction, a fluid, such as sterile saline solution, is infused in and around the cutting tool 56 in a continuous micro flow that can measured in millimeters of solution per minute (e.g., “Fluid Flow”). The Fluid Flow builds up in the transfer tool 34 to form the “liquid piston” directly underneath the pin 58 (e.g., between the pin 58 and the tissue). The cutting tool 56 slips down the liquid column based on the pressure from the Fluid Flow and controlled by the force profile. The pressure is sufficient to break through the surface of the tissue and capture an MTC 12 as a solid mass within the tissue space 62 of the cutting tool 56. In addition to limiting the cellular damage to the MTC 12 due to dehydration, the Fluid Flow protects the MTC 12 as part of the liquid piston, reducing both the compression of the MTC 12 and the forces pulling the MTC 12 apart.

The force profile informs control of the Z-axis change for lifting up the transfer tool 34 to extract the MTC 12. The pin 58, the cutting tool 56, and the liquid piston surrounding the individual MTC 12 all come up together above the surface 23 of the tissue, based on the parameters of the force profile, to complete the extraction. Accordingly, the result of step 96 is a fractional skin graft that includes an individually harvested MTC 12. As described above, rather than a single, large donor site wound, the fractional skin grafting techniques described above create minor donor site wounds that can heal with minimal to no scarring.

Referring now to step 98, the extracted MTC 12 can be oriented and extruded into a matrix 16 to form any shape for the graft 20, restrained only by the programing limitations of the system 10. More specifically, the MTCs 12 are extruded from the transfer tool 34 to the supportive matrix 16 at desired locations, resulting in the custom graft 20. For example, harvested MTCs 12 are at least assembled in a desired orientation matching an epidermal-dermal polarity of normal skin. Furthermore, harvested MTCs 12 can be spaced relative to each other to match their original spacing at the donor site 14. Alternatively, in some applications, the MTCs 12 can be oriented to decrease a spacing between MTCs 12 compared to their original spacing when extracted from the donor site 14.

In some applications, a graft assembly station, housing the graft construction site 21, can be located within operational range of the system 10 at the time of MTC harvesting (e.g., adjacent the donor site 14 or the recipient site 18). Thus, the completion of step 98, resulting in a custom graft 20, may not directly involve the recipient site 18. Rather, the transfer tool 34 can be moved (e.g., by control of its X, Y and/or Z movement) to a separate biocompatible substrate 16 of any shape located in the graft assembly station. The MTCs 12 can be extruded into the matrix 16 (e.g., a gelatinous or fibrous substrate material) to hold the oriented MTCs 12 temporarily in place until the graft 20 is complete. MTC extrusion from the transfer tool 34 into the supportive matrix 16 relies on the Fluid Flow and is controlled by the Force Profile, e.g., in reverse of that described above.

If the graft 20 is incomplete (i.e., it requires additional MTCs 12), as determined at step 100, steps 96 and 98 can be repeated to further harvest, extract, and extrude all MTCs 12 into a complete custom graft 20. The completed custom graft 20 thus consists of both the biocompatible substrate 16 and the build-up of MTCs 12 placed as determined by the MTC Skin Construct Plan.

As described above, the supportive matrix 16 (e.g., a supportive biomaterial such as a collagen solution or biocompatible matrix) is used to orient the MTCs 12 and/or maintain MTC orientation in a construct. More specifically, the supportive matrix 16 can be used to create a graft 20 that maintains the overall structure and orientation of the assembled tissue columns 12. This results in a more easily handled graft 20 and, in some applications, can allow for physicians to add drugs, other components, or other cell types as needed to the graft 20.

Accordingly, the supportive matrix material 16 may be biocompatible so that the entire completed custom graft 20, including the matrix material, may be placed directly into a wound of the recipient site 18 (in accordance with step 102, as further described below). Example biocompatible matrices include, but are not limited to, decellularized tissue (e.g., skin, gut, amnion, or other tissue that has been processed to remove all living cells, so all that's left of the original tissue are the extracellular components), matrices made from natural biomolecules (collagen, fibrin, hyaluronan, etc., used alone or in combination) in various forms (e.g., in a gel or spun into fibers), synthetic materials that are biodegradable and have certain bio-mimicking properties (e.g., biodegradable polymers functionalized with cell adhesion moieties), and matrices including collagen, hydrogels, fibrin gels, or carbon scaffolds. Additionally, any of the above examples can include growth factor and/or oxygen concentration enhancing material (e.g., CaO2) and/or other substances.

Referring back to the method of FIG. 5, once the custom graft 20 is complete, as determined at step 100, the graft 20 can be applied to a recipient site 18 (such as a wound) at step 102. More specifically, following steps 92 through 100, a three-dimensional, full-thickness graft 20 is available for wound healing, and this graft 20 includes MTCs 12 in substantially vertical, epidermal-dermal orientation. Such a graft 20 is three-dimensional because it has a usable width, length, and height and is full-thickness because is includes at least epidermal and dermal layers 22, 24. In some embodiments, a custom graft 20 may be round. However, in other embodiments, a custom graft 20 may be rectangular, square, or another suitable shape.

According to step 102, the graft 20 can be applied to the recipient site 18 as any other type of full-thickness tissue graft. For example, the graft 20 can be placed in or on a wound in order to entirely, or at least partially, cover the wound. Alternatively, the completed custom graft 20 can be stored in a sterile environment for a time period before being transferred to the recipient site 18.

In light of the above, the present methods allow for assembling multiple MTCs 12, in a desired orientation, into solid, three-dimensional tissue grafts 20 with precise shape and depth based on digitized characteristics of the recipient site 18. Furthermore, a system 10 may be provided to fully execute the above-described methods 70, 90. When the graft 20 is applied to a recipient site 18, the full-thickness MTCs 12 can grow, complete with sweat glands and other complex features of the harvested tissue. Accordingly, these MTCs 12 can be used to assist and improve tissue healing at the recipient site 18 (such as a wound). More specifically, properly oriented MTCs 12 can improve healing by accelerating re-epithelialization processing, recapitulating normal dermal architecture, and/or reducing scarring, as compared to healed untreated wounds and healed wounds treated with randomly oriented MTCs 12.

In particular, while harvested MTCs 12 can be applied to wound beds randomly, that is, without maintaining the normal epidermal-dermal polarity of skin, MTCs 12 organized in a defined epidermal-dermal orientation can be advantageous to accelerate wound healing by providing for more efficient cell and tissue growth and more faithful replication of normal tissue microanatomy (for example, complex structures in full-thickness tissue grafts, like hair follicles, have defined polarities and are generally less tolerant of being implanted in the wrong orientation). Thus, while randomly oriented MTCs 12 have been shown to improve healing compared to untreated wounds (e.g., by healing faster with less contraction), MTCs 12 assembled and oriented in accordance with the systems and methods described above can further improve healing time, contractile response, skin appearance, and/or structural organization.

In light of the above, small columns of full-thickness skin tissue can be harvested, with each donor wound being small enough to heal quickly by regeneration with minimal to no scarring. While such columns can be applied to wound beds randomly to accelerate wound healing, using tissue columns organized in a defined epidermal-dermal orientation can be advantageous by providing for more efficient cell and tissue growth and more faithful replication of normal tissue microanatomy. That is, in comparison to randomly oriented MTCs, MTC grafts arranged in an epidermal-dermal orientation can provide faster healing time with less contractile response, and result in a healed wound that better matches normal tissue coloring and structure (e.g., that better matches an appearance and structure of the tissue that surrounds the recipient site). Furthermore, the above methods and systems for grafting and assembling MTCs are simple and nontoxic, using biocompatible supportive materials, and permit grafts to be built in an iterative manner, thus permitting harvesting of MTCs into a custom graft capable of properly fitting a desired size and geometry of a recipient site.

The above methods and systems may be used in different wound healing applications, such as, but not limited to, burns, abrasions, and surgical wounds, or other grafting applications, such as, but not limited to, vitiligo. Additionally, while the above methods and systems have been described with respect to skin grafts, the principles described herein may applied to other tissue types as well. For example, the above methods and systems may be used with other types of tissue, such as, but not limited to, tissue of the liver, kidney, or heart, to provide micro tissue columns arranged in a desired orientation.

The present invention has been described in terms of one or more preferred embodiments, and it should be appreciated that many equivalents, alternatives, variations, and modifications, aside from those expressly stated, are possible and within the scope of the invention. Furthermore, the term “about” as used herein means a range of plus or minus 20% with respect to the specified value, more preferably plus or minus 10%, even more preferably plus or minus 5%, most preferably plus or minus 2%. In the alternative, as known in the art, the term “about” indicates a deviation, from the specified value, that is equal to half of a minimum increment of a measure available during the process of measurement of such value with a given measurement tool. As used herein, unless otherwise specified or limited, “at least one of A, B, and C,” and similar other phrases, are meant to indicate A, or B, or C, or any combination of A, B, and/or C. As such, this phrase, and similar other phrases can include single or multiple instances of A, B, and/or C, and, in the case that any of A, B, and/or C indicates a category of elements, single or multiple instances of any of the elements of the categories A, B, and/or C.

1. A system for assembling a plurality of micro tissue grafts into a custom graft, the system comprising:
a pick and place unit including a moveable arm;
a transfer tool coupled to a distal end of the moveable arm; and
a control unit configured to:
control the moveable arm to move the transfer tool to a donor site and a graft construction site, and
control the transfer tool to harvest individual micro tissue grafts from the donor site and arrange the micro tissue grafts into a supportive matrix to form the custom graft.

2. The system of claim 1, wherein the transfer tool includes a hydroextractor configured to harvest one of the plurality of micro tissue grafts.

3. The system of claim 2, wherein the hydroextractor includes a hollow cutting tool, a pin, and a liquid delivery system, wherein the hollow cutting tool is sized to pierce the donor site to surround one of the plurality of micro tissue grafts.

4. The system of claim 3, wherein the liquid delivery system is configured to create a column of fluid surrounding the one of the plurality of micro tissue grafts to create a hydraulic coupling between the one of the plurality of micro tissue grafts and the hollow cutting tool.

5. The system of claim 3, wherein the liquid delivery system is configured to supply pressure between the hollow cutting tool and the pin to maintain the one of the plurality of micro tissue grafts within the hollow cutting tool.

6. The system of claim 5, wherein the liquid delivery system is configured to reverse the pressure supplied between the hollow cutting tool and the pin to extrude the one of the plurality of micro tissue grafts into the supportive matrix.

7. The system of claim 3, wherein the hydroextractor is configured to use fluid pressure to lift the one micro tissue graft, surrounded by the hollow cutting tool, from the donor site.

8. The system of claim 3, wherein the pin is slidably received within the hollow cutting tool.

9. The system of claim 3, wherein the transfer tool includes a linear actuator coupled to the hollow cutting tool.

10. The system of claim 9, wherein the linear actuator is configured to displace the hollow cutting tool relative to the pin.

11. The system of claim 1 and further comprising one or more image sensors configured to obtain an image of a recipient site.

12. The system of claim 11, wherein the control unit is configured to receive the image of the recipient site from the image sensors and create a wound profile of the recipient site, based on the image, that defines a depth and shape of the custom graft.

13. The system of claim 11, wherein the one or more image sensors are further configured to obtain an image of the donor site.

14. The system of claim 13, wherein the control unit is configured to receive an image of the donor site from the image sensors and create a construct plan of the donor site, based on the image, that maps the plurality of micro tissue grafts.

15. A method for assembling a plurality of micro tissue grafts, the method comprising:
a) analyzing a recipient site by imaging the recipient site and creating a wound profile;
b) selecting a donor site based on the wound profile;
c) individually harvesting each the plurality of micro tissue grafts from the donor site; and
d) individually arranging each the plurality of micro tissue grafts in a substantially vertical epidermal-dermal orientation within a supportive material to create a custom graft.

16. The method of claim 15 and further comprising applying the custom graft to the recipient site.

17. The method of claim 15, wherein step c) includes creating a hydraulic coupling between each of the plurality of micro tissue grafts and a hydroextractor.

18. The method of claim 15, wherein creating a wound profile includes defining a shape and depth of the custom graft.

19. The method of claim 18, wherein step b) includes creating a construct plan of the donor site that maps the plurality of micro tissue grafts within the donor site.

20. The method of claim 15, wherein step b) includes creating a construct plan of a plurality of potential donor sites and comparing the construct plans to select the donor site.

21. The method of claim 15, wherein the supportive material is a biocompatible matrix.

22. A method for assembling a plurality of micro tissue grafts using a system including an arm, a transfer tool, and a sensor, the method comprising:
a) creating a wound profile by positioning the sensor over a recipient site and imaging the recipient site;
b) positioning the sensor over a donor site to map the plurality of micro tissue grafts within the donor site based on the wound profile;
c) individually extracting one of the plurality of micro tissue grafts from the donor site into the transfer tool using hydraulic pressure;
d) individually extruding the one micro tissue graft from within the transfer tool into a supportive material; and
e) repeating steps c) and d) to create a custom graft.

23. The method of claim 22 and further comprising continuously bathing the one micro tissue graft in a fluid when it is extracted into the transfer tool.

24. The method of claim 22, wherein step d) includes reversing the applied hydraulic pressure from step c).

25. The method of claim 22 and further comprising applying the custom graft to the recipient site.

26. A method for harvesting a micro tissue graft from a donor site to into a supportive material using a hydroextractor, the method comprising:
contacting a surface of the donor site with the hydroextractor so that a lower surface of a hollowing cutting tool and a lower surface of an internal pin of the hydroextractor contact the surface of the donor site;
delivering a liquid between the pin and the hollow cutting tool so that the liquid travels to the surface of the donor site creating a hydraulic coupling between the hydroextractor and the surface;
moving the hollow cutting tool downward into the tissue while the pin remains flush with the surface to harvest the micro tissue graft within a tissue space of the hollow cutting tool, the liquid sealing the micro tissue graft within the tissue space;
retracting the hydroextractor away from the surface of the donor site to extrude the micro tissue graft from the donor site;
contacting a surface of the supportive material with the hydroextractor so that the lower surface of the hollow cutting tool contacts the surface of the supporting material;
lowering the hydroextractor into the supporting material until the pin is level with the surface of the supporting material; and
retracting the hollow cutting tool from the supporting material so that the micro tissue graft remains within the supporting material.
